(19) United States
(12) Patent Application Publication
(10) Pub. No.: US 2016/0054297 A1
(43) Pub. Date: Feb. 25, 2016

(54) METHOD AND APPARATUS FOR DETECTING BREATH ALCOHOL CONCENTRATION BASED ON ACOUSTIC BREATH SAMPLER

(71) Applicant: 2045 Tech S.R.L., Caorle (IT)

(72) Inventors: Marco Barbetta, Piove Di Sacco (IT); Fabio Penzo, Caorle (IT)

(21) Appl. No.: 14/856,888

(22) Filed: Sep. 17, 2015

Related U.S. Application Data
(63) Continuation of application No. PCT/EP2013/055883, filed on Mar. 21, 2013.

(51) Int. Cl. G01N 33/497

(52) U.S. Cl. CPC G01N 33/4972

(57) ABSTRACT

An apparatus for detecting gaseous components in a breath, including a breath channel for receiving breath, a gas sensor and a flow sampler made with a vortex whistle flow-meter. Measuring the volume passed through the breath channel, and recovering the gaseous component concentration from the gas sensor reading and from the sampler reading.

### CROSS REFERENCE TO RELATED APPLICATIONS

This application is a continuation of PCT Application No. PCT/EP2013/055883, filed on Mar. 21, 2013. The contents of this application are incorporated by reference herein, in their entirety and for all purposes.

### TECHNICAL FIELD

The present invention relates to the field of detectors of gas concentration in breath, comprising both devices and methods. In particular, the present invention relates to portable and/or fixed breath alcohol concentration detectors. Such devices allow estimating the blood alcohol concentration indirectly by measuring the gaseous alcohol concentration in breath. Some of the devices belonging to this category are named Breathalyzer™, which is a trademark of Draeger Safety, A.G.

### BACKGROUND OF THE INVENTION

The detection of blood alcohol concentration is useful to estimate a person's sobriety and to determine if such person is able to drive. Many methods for measuring blood alcohol concentration are based on an indirect measurement. The measurement is performed on breath, and the gaseous ethanol concentration in breath (BrAC) is measured and then the blood alcohol concentration (BAC) is recovered by means of a well-known relation.

Methods for measuring gas ethanol concentration can be based on infrared spectroscopy, semiconductor sensors or fuel-cell sensors. All of them sample a part of the exhaled gas through a mouthpiece.

The first method brings accurate results, but is neither scalable to a portable device nor cost-effective for a consumer application.

The second method is less expensive, but quite inaccurate and needs electric power to be delivered to heating elements for the sensors, which makes this method less effective for ultra-low power portable devices.

An example of implementation of this second method is described in EP1046910. The apparatus uses a semiconductor ethanol sensor, which the gas blown by the user is delivered to.

The semiconductor sensor is sensible to ethanol when the sensing part is heated to high temperature with an electronic resistor heater. However, the amount of power needed by the heated sensor (typically more than 50 mW) could be excessive for certain low power applications, such as button-cell powered keychain or externally powered devices or power-harvesting devices. The third method, using fuel-cell sensors, has the advantage to be both accurate and ultra-low power, as the fuel-cell element usually needs no heating elements. The sensing is performed by delivering a known volume of gas to a chamber which contains the fuel cell element and measuring the cell current output. As the fuel-cell output is dependent on the volume of gas flowed through the chamber, this has to be known. Methods for knowing a priori the volume consist in an active sampling system that pumps a fixed volume of gas inside the chamber with an electro-mechanical device. The power consumption and complexity of such system make it less effective for portable consumer devices if compared to other solutions.

Methods for knowing a posteriori the volume consists on a passive flow-meter which measures the volume passed through the chamber, and sometimes is called pump-less method. This method has been implemented sometimes with a thermal flow-meter. This has the issues of needing a certain amount of power for self-heating (typically more than 10 mW), and generally has a poor response time and its accuracy depends on environmental conditions. This method has been implemented other times with a pressure-based flow-meter, with a calibrated orifice plate or a Venturi tube. These methods have the issues of being more complex to calibrate and less cost-effective.

An example of implementation of the third method, but related to spirometry measurement, is described in EP0437055, where a tube containing a diameter constriction is provided. The pressure difference between upstream and downstream tube is strictly dependent on fluid flow rate inside the tube. A differential pressure sensor is used to recover the flow measurement. To the skilled person it is immediately apparent that the problem of measuring a high volume of gas is quite different than measuring a small volume of gas, in which a small concentration of a specific substance must be detected and that the solution of the first problem is not applicable to the solution of the second one.

Another example of implementation of the third method is described in EP0834072. In the apparatus described, a pump is used to sample a predetermined gas volume from the main pipe while the user is blowing. This volume, when the pump is activated, passes through the chamber of a fuel-cell ethanol sensor with which the pump is in communication. This assures a known volume into the sensor, but the pump itself needs an amount of electrical current (typically more than 0.5 A) which, even if needed impulsively, could be excessive for certain applications. Moreover, the implementation of such electro-mechanical pump could not be cost-effective for some applications.

Another example of implementation of the third method, which does not use a sampling pump, is also described in U.S. Pat. No. 6,026,674. The apparatus described uses a pressure sensor to detect the presence of breath flow and an electro-mechanical valve to deliver an amount of gas volume to the fuel-cell. In this implementation, the exact gas flow is not measured, but the dosing is demanded to the valve timing. Moreover, in this implementation, the power consumption of the electro-mechanical valve could be excessive for certain applications.

Another example of implementation of the third method, which does not use a sampling pump, is also described in U.S. Pat. No. 3,966,579. A fraction of the breath flow is passed through the fuel-cell and its short-circuit current is measured. However, a method to measure the sample volume in order to retrieve the concentration is not described.

Another example where a sampling pump is not used is described in US2011/0009765. The apparatus described uses a thermistor, possibly heated, to measure the flow rate in the main pipe and so to estimate the volume. The flow transfers heat to or from the thermistor, thus altering its temperature in a way that is dependent on the flow rate. Specific information on the accuracy of the flow measurement is not given in the document. Moreover, the power needed to heat the sensor could be excessive for certain applications.

An example of a method used for data processing inside an implementation of the third method is described in WO2012/087186, wherein the flow rate detected is continuously integrated to retrieve the total volume of sampled fluid, and this information is then used to compensate the signal from the ethanol sensor. The document aims to solve accuracy of the measurement and solves the problem through an improved mathematical method.

Power harvesting devices are devices that exploit a power source not intended by design to supply power to a device. Examples are moving objects or bodies and audio ports for earphones, headphones or loudspeakers.

An example of implementation of a power harvesting device using audio port is described in ACM DEV′10, Dec. 17-18, 2010 (978-1-4503-0473-3-10/12), wherein a jack audio connector is connected to an electric transformer and a diode rectifier bridge, so that the alternating current (AC) voltage from the audio port is increased and converted to direct current (DC) voltage to power the device. In the document it is declared that the maximum power drawn from the audio port is less than 20 mW.

There is still the need for an ultra-low power yet precise measurement system for gaseous ethanol concentration that can be cost-effective for a portable device, therefore based on a sampling system having the aforementioned advantages.

### SUMMARY OF THE INVENTION

It has now been found a solution to the above-mentioned problem of providing a precise and accurate sampling system which needs ultra-low power consumption by using a vortex flow-meter as sampling system.

An example of implementation of the vortex flow-meter used in some of the embodiments of the present invention is described in U.S. Pat. No. 2,794,341. The flow-meter is used for measuring air speed in airplanes, water speed in ships, pressure in tubes, or as a musical toy. In particular the apparatus described in FIG. 2 of the mentioned document is suitable for the present invention.

The working principle of the vortex flow-meter (also known as vortex whistle) is described in the present document, but can be further understood in IEEE S 0018-9456(00)02236-1 (Sato, H. et al.) and in SICE TR 0007/99/3507-0840 (Sato, H. et al.).

An object of the present invention is an improved device for detecting and determining concentration of a gaseous substance in a person's breath, in particular ethyl alcohol. The advantages of the invention are a very low-power consumption, low-cost and small size, but preserving a high accuracy on the sampled volume, resulting on an improved precision on the concentration measurement with respect to thermal flow-meter-based samplers. In terms of precision, the present invention can be related to samplers based on calibrated orifice or Venturi-tube, but presents many advantages in terms of construction simplicity and costs.

The present invention is applicable to a sensor system in all cases in which the sensor reading is dependent on the sampled volume, and so this volume has to be known.

It is an object of the present invention an apparatus for detecting gas concentration in exhaled breath of a subject, comprising a sampling device, a detector and a display device, characterized in that said sampling device is a vortex flow-meter.

Within the meanings of the present invention, said apparatus is also briefly named “breath analyser”.

Advantageously, said apparatus can be portable or fixed.

In a preferred embodiment, in the apparatus according to the present invention said detected gas is ethanol, preferably, said detected ethanol concentration is directly correlated to ethanol blood concentration in said subject.

In a preferred embodiment of the present invention, breath or blood ethanol concentration in said subject is displayed.

In a preferred embodiment of the present invention, in said apparatus, said detector is a fuel-cell.

In a preferred embodiment of the present invention, said apparatus comprises:
a. an assembly 15, wherein there is provided a main breath channel 1 for breath sampling, said main channel 1 is connected in parallel with a secondary channel 2, so that a portion of the main flow of the sampled breath passes through the secondary channel 2 and returns to the main channel;
b. an ethanol sensor 3 placed in the secondary channel, so that said portion of the sampled breath passes through said sensor, said sensor can optionally be equipped with a temperature sensor;
c. a vortex flow-meter 5 placed at the end of the main channel 1 to collect the sampled breath;
d. a transducer 7 located in the vortex flow-meter 5 to obtain an electrical signal;
e. two amplifiers 8, 9 connected by way of input to the sensor 3 and to the transducer 7, respectively and by way of output to a microprocessor with a memory containing a software algorithm 10 for the calculation of ethyl alcohol concentration;
f. a display 12 connected to the microprocessor 10.
Alternatively, instead of the item a) above, in said assembly 15 there is provided:
a′. a main breath channel 1 for breath sampling, said main channel 1 is connected in series with said vortex flow-meter 23, from which a first tangential pipe 41 connected to the said vortex flow-meter 23 and to the sensor 43, and then reconnected to the said vortex flow-meter 23 by a second tangential pipe 42.

In different embodiments of the present invention, in said apparatus said transducer 7 is a microphone or a piezoelectric transducer.

In the apparatus according to the present invention, power is provided by at least one battery or by an external power source. Therefore, a power channel 11 is provided to supply the necessary power to the device. Conveniently, the power channel 11 can also be a communication channel to receive and transmit data to and from the device, for example in case of a wired channel. Channel 11 can be a communication channel only. For example, in case of wireless, the communication channel 11 is coupled with a power supply. Also in case of a wired communication channel, an external power supply should be provided.

In a preferred embodiment, in said apparatus, said communication and power channel 11 is a Universal Serial Bus connection, preferably said communication channel 11 is a wireless connection for communication and a battery for power.

In a more preferred embodiment, in said apparatus said communication and power channel 11 is an audio jack connector.

In another embodiment, said communication channel is a jack connector and power is supplied by a separate battery.

In another preferred embodiment, said apparatus further comprises an intelligent device with display 13 connected to the microprocessor 10 instead of the display 12.

Said intelligent device 13 is selected from the group consisting of a smartphone, a laptop and a desktop personal computer.

In another preferred embodiment, said apparatus further comprises a vehicle interlock system 14 connected to said microprocessor 10.

In a preferred embodiment, said apparatus is connected with said intelligent device 13 or said vehicle interlock system 14 with a data communication channel 11, both in a wired or wireless mode.

Another object of the present invention is a method for detecting breath alcohol concentration (BrAC) and subsequently, if desired, converting said blood alcohol concentration (BAC) in a subject comprising sampling a subject's breath into the above apparatus.

According to the method of the present invention, the exhaled breath sampled from said subject produces an acoustic wave in said vortex flow-meter, said acoustic wave having a frequency f and the method comprises:
a. converting said frequency f into a flow rate F;
b. calculating from said flow rate F the total volume V of said sampled breath;
c. integrating current I from said detector to obtain the total charge Q;
d. calculating BrAC according to equation (4)

$$BrAC = K \cdot \frac{Q}{V}$$

wherein K is a calibration parameter;
e. optionally converting BrAC into BAC according to equation (6).

BAC=K′·BrAC

The above mentioned apparatus can be incorporated in a vehicle interlock system 14, which can be mounted on a vehicle. Both the system and the vehicle are objects of the present invention, together with a method for controlling the operation of a vehicle provided with said vehicle interlock system 14 in function of the blood alcohol concentration of the vehicle operator, said method comprising:
a. determining blood alcohol concentration of said vehicle operator, by sampling said operator's breath into said system;
b. comparing said blood alcohol concentration with a determined alcohol concentration set into said interlock system;
c. operating said interlock system, in order to prevent the user to ignite the vehicle, if said blood alcohol concentration is equal or higher than said determined alcohol concentration.

A method for operating the apparatus herein disclosed is another object of the present invention and said method comprises:
a. placing the apparatus in the START condition 51;
b. providing an exhaled breath sample for a certain amount of time, in which sampler data, in form of the detected frequency f are collected 52;
c. converting frequency f into flow rate F by inverting equation (1) f=k·F;
d. passing said apparatus to a decisional condition 53 in which, if the flow F is greater than a predefined threshold F₂, the apparatus passes to the subsequent condition 54, otherwise it goes back to condition 52;
e. in the condition 54, both the vortex flow-meter sampler and the sensor are sampled for a certain amount of time;
f. passing said apparatus to condition 55 in which the acquired flow data is integrated to obtain the total volume data according to the equation (2)

V(nT)=Σₖ₌₁ⁿF(kT)·T
in which F(kT) is the flow value recovered with equation (1) from the frequency f measured in the k-th sampling window, T is the length of the sampling window, V(nT) is the volume summed from the first to the n-th sampling window, and the first sampling window is taken when the condition 53 is first verified, and so determines the beginning of the flow;
g. passing said apparatus to decisional condition 56 in which, if the volume V is greater than a predefined threshold V₁, the apparatus passes to the condition 58, otherwise it goes to the condition 57. In this decisional condition, if the flow F is greater than a predefined threshold F₁ the apparatus goes back to the condition 54 in which it continues to sample and integrate the two values, otherwise it passes to the FAIL condition 62;
h. in the condition 58, acquiring detector current I and integrating it to obtain the total charge Q according to equation (3)

Q(nT)=Σₖ₌₁ⁿI(kT)·T
in which I(kT) is the sensor current measured and optionally averaged in the k-th sampling window, T is the length of the sampling window, Q(nT) is the charge summed from the first to the n-th sampling window, and the first sampling window is taken when the condition 53 is first verified, and so determines the beginning of the flow;
i. passing said apparatus to condition 59 in which the BrAC is calculated according to equation (4)

$$BrAC = K \cdot \frac{Q}{V}$$

in which K is a calibration parameter, Q is the charge obtained from equation (3) and V is the volume obtained from equation (2); the apparatus then passes to the condition 60 in which the BrAC result is displayed, or transmitted or stored for further usage; the measurement process then concludes to the END condition 61;
j. displaying BrAC.

The present invention will be now disclosed in detail also by means of drawings and examples.

### BRIEF DESCRIPTION OF THE DRAWINGS

While the following paragraphs will describe the invention in details, it is believed that such details can be better understood with the aid of the drawings in which:

FIG. 1 shows the setup of a breath ethanol concentration detector device according to the first embodiment of the invention, considering only some elements, or to the fourth or fifth embodiments of the invention, considering different elements;

FIG. 2 shows in detail the shape of the vortex flow-meter as long as the positioning of the microphone and the piezoelectric transducer;

FIG. 3 shows the tangential air dump system according to the second embodiment of the invention;

FIG. 4 shows a flow chart of the measurement method for an apparatus including an intelligent device with display, according to the fourth embodiment of the invention, or for an apparatus according to the fifth embodiment of the invention.

FIG. 5 shows a flow chart of another measurement method for an apparatus including an intelligent device with display, according to the fourth embodiment of the invention, or for an apparatus according to the fifth embodiment of the invention.

### DETAILED DESCRIPTION OF THE INVENTION

The present invention is now disclosed in detail through some preferred embodiments. In this connection, the embodiments are provided in the exemplary working of the invention for the detection of ethyl alcohol concentration in the exhaled breath of a subject and the subsequent determination of his/her ethyl alcohol concentration in blood. As an exemplary embodiment, the detection of ethyl alcohol is made by means of a fuel-cell sensor.

For the purposes of the present invention the terms “system”, “device” and “apparatus” are meant as synonyms.

For the purposes of the present invention the terms “ethyl alcohol”, “ethanol” and “alcohol” are meant as synonyms.

For the purposes of the present invention the terms “user”, “subject”, and “person” are meant as synonyms.

When a breath sample is passed through the chamber of a fuel-cell ethanol sensor, an electrochemical reaction takes place together with an electrolyte solution inside the sensor, and an electric current can be measured at the sensor electrodes.

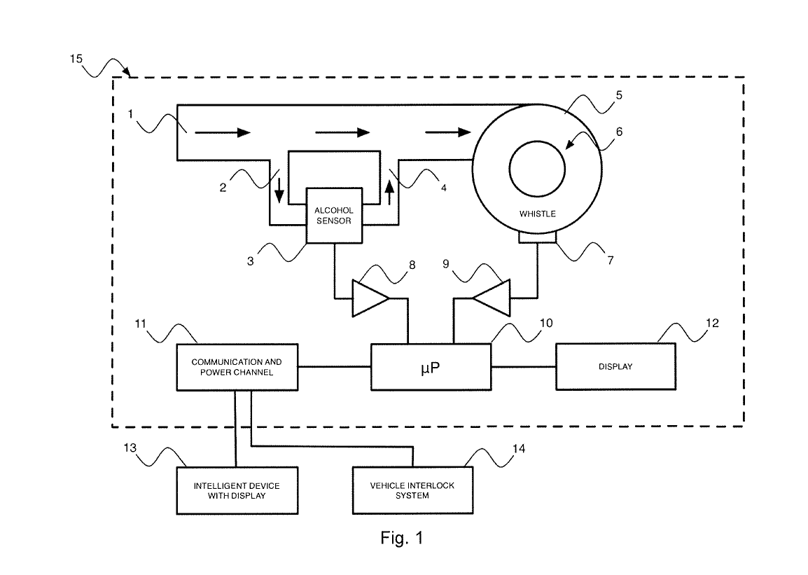

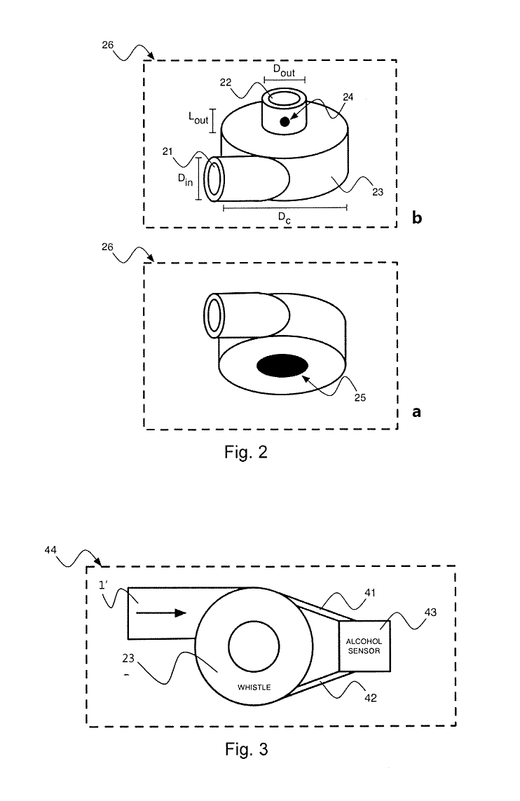

According to a first embodiment of the invention (see FIG. 1) in the above apparatus, there is provided an assembly 15, comprising a sampling device (also called whistle), a detector 3 (also called sensor, and for this specific first embodiment of the invention is an ethyl alcohol sensor), and a display device 12 is shown. In the sampling device, there is provided a breath channel 1, called the main breath channel. The arrows show the flow direction of the sampled breath from the subject whose blood alcohol concentration is to be measured. The main breath channel 1 is connected in parallel with a secondary channel 2, 4. A portion of the main flow passes through the secondary channel 2 and returns to the main channel. An ethanol sensor 3, for example a fuel-cell sensor, is placed in the secondary channel. A vortex flow-meter 5 is placed at the end of the main channel 1. The vortex flow-meter 5 is composed of a cylindrical chamber with a tangential inlet and an axial outlet 6, such as for example shown in the above mentioned U.S. Pat. No. 2,794,341, FIG. 2. A microphone or piezoelectric transducer 7 is located in the vortex flow-meter 5 to obtain an electrical signal. Two amplifiers 8, 9 are connected by way of input one to the sensor 3 and one to the microphone or transducer 7 and by way of output to a microprocessor with a memory containing a software algorithm 10 for signal processing and the calculation of ethyl alcohol concentration. A display 12 is connected to the microprocessor 10.

The system according to the present invention can optionally be connected to an intelligent device with display 13, such as for example a smart-phone, instead of the display 12.

The system according to the present invention can optionally be connected to a vehicle interlock system 14.

In a second embodiment of the invention, as far as the vortex flow-meter is concerned, and as shown in FIG. 3 the secondary flow is obtained in an alternative way, by means of two tangential pipes connected to the vortex flow chamber. The two pipes are then connected to the ethanol sensor.

In a third embodiment of the invention, a data communication channel, wired or wireless is added to the microprocessor system.

In a fourth embodiment of the invention, a data communication channel connected to the microprocessor system and to an intelligent device (smart-phone) with display and user interface is added to the system. The raw flow and current data and/or the calculated measurement are transmitted to the intelligent display device. The smart-phone can optionally execute an application that communicates with the device in order to display, elaborate and share the data collected from the device. The application can optionally represent on the smartphone screen the BAC and/or BrAC measured.

The application can optionally calculate and display the “recovery time”, which is the time by which the user's BAC is expected to fall below a certain selected limit. The calculation can be possibly made accounting for the user's physical characteristics. Examples of these implementations can be found on applications such as “AlcoDroid Alcohol Tracker” by Myrecek or “DrinkTracker” by slappmedotcom Pty Ltd.

In a fifth embodiment of the invention, a data communication channel connected to the microprocessor system and to an interlock system for a vehicle is added to the system.

Reference will now be made in detail to the present exemplary embodiments of the invention, examples of which are illustrated in the drawings, in which each number indicates a unique element.

Referring to FIG. 1, the assembly 15 has a main flow channel made of a main pipe 1 with an inlet for receiving the user's breath. The end of the main pipe is connected to the inlet of a vortex whistle flow-meter 5 which has an outlet 6 to allow the flow to be expelled. The main pipe has a lateral derivation 2 connected to an ethanol sensor 3 which is then connected to another derivation 4 that reconnects to the main pipe. In this way, the airflow inside the sensor is proportional to the flow in the main pipe. The ethanol sensor output is an electrical current. The output of the vortex flow-meter is an electrical voltage audio signal delivered by an acoustic transducer 7. The output of the ethanol sensor and the whistle are electrical signals and are connected to two amplifiers 8 and 9 that are then connected to a microprocessor unit with memory 10.

Referring to FIG. 2a,b, the vortex flow-meter 26 is provided with a tangential inlet 21, a round chamber 23 and an axial outlet 22. The airflow entering into the chamber produces an instable fluid dynamic condition that results in an oscillation which frequency f is linearly proportional to the input flow F according to equation (1).

f=k·F (1)

wherein k is a calibration constant which is stored for example in the microprocessor memory.

The oscillation produces an acoustic wave at the same frequency. The acoustic wave can be measured with the transducer 7. In an exemplary embodiment of the invention, the transducer 7 is a microphone 24 positioned on the axial outlet whereas in another exemplary embodiment it is a piezoelectric transducer 25 positioned on the bottom wall, either on the outside or on the inside face.

Always referring to FIG. 2b, it has been found that a determined interval of mechanical dimensions of the vortex flow-meter 26 makes it compatible with the dynamic range of the flow rate of the user's blowing (5-15 L/min). Such dimensions grant a good signal-to-noise ratio (SNR) over the said dynamic range and mitigate intrinsic nonlinearities. In a preferred embodiment of the vortex flow-meter 26 such dimensions are:
Dᵢₙ from 4 to 7 mm,
Dₒᵤₜ from 6 to 9 mm,
D_{c} from 25 to 35 mm;
Lₒᵤₜ from 4 to 15 mm;
   wherein Dᵢₙ represents the diameter of the tangential inlet 21, Dₒᵤₜ represents the diameter of the axial outlet 22, D_{c} represents the diameter of the chamber 23 and Lₒᵤₜ represents the length of the axial outlet 22.

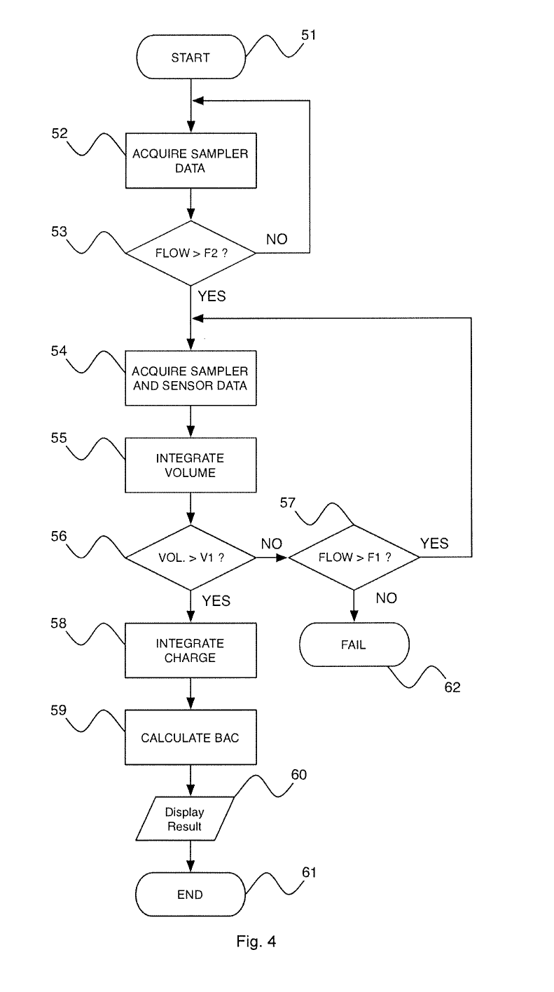

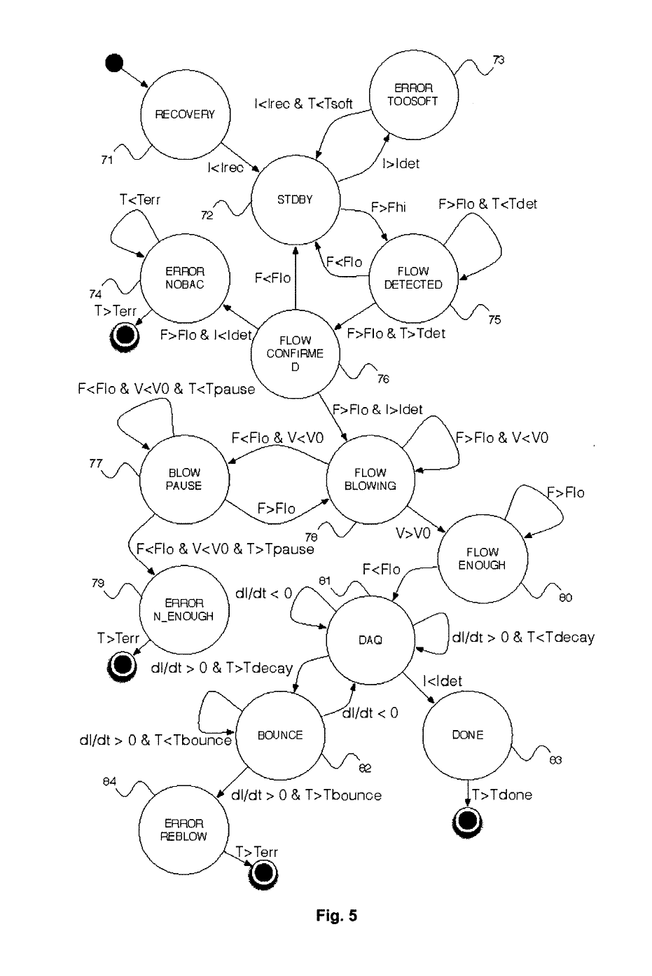

In the first embodiment of the invention, the microprocessor is connected to a display 12. The microprocessor 10 loads the application code, made according to the method described in FIG. 4 or FIG. 5, from its memory. The signal from the amplifier 9 connected to the flow-meter is pre-elaborated by the microprocessor in order to recover the frequency f, which is then used as the first input for the algorithm. The signal from the amplifier 8 connected to the ethanol sensor is used to recover the sensor current I, which is used as the second input for the algorithm. After performing the algorithm, the calculated BrAC (see equation (4) below, or an equivalent algorithm, such as for example WO 2012/087186) or BAC (see equation (5) below) result is displayed on the display 12, which is connected to the microprocessor.

Optionally, in the case of embodiments comprising an intelligent display device (fourth) or vehicle interlock (fifth) the algorithm in FIG. 4 or FIG. 5 can be implemented totally on these devices and the microprocessor only instructed to acquire data and perform signal processing to recover f. Each intermediate solution, where parts of the algorithms in FIG. 4 or FIG. 5 are implemented on the microprocessor and complementary parts implemented on the intelligent devices, are also comprised in the present invention.

In one exemplary embodiment of the invention the assembly 15 can be powered by batteries or external power.

In a second embodiment of the invention, the assembly 15 is modified in the components 2, 4, 5, shown in FIG. 1, according to the setup 44 described in FIG. 3. The lateral derivation (i.e. the secondary channel) is not realized on the main pipe but on the vortex chamber 23. A first tangential pipe 41 is connected to the vortex chamber and to the sensor 43, and is then reconnected to the chamber by a second tangential pipe 42. In this way part of the airflow is spilled out of the chamber and then back injected in it. In an opportune interval, the flow passing through the sensor is proportional to the flow entering in the device. This provides the same performance of the first embodiment of the invention, but reducing the occupied space.

In the third embodiment of the invention, the communication and power channel 11 is used both for powering the device and transfer the acquired or calculated data from the method in FIG. 4 or FIG. 5. In an exemplary embodiment of the invention, the communication channel and power channel 11 is a Universal Serial Bus (USB) connection. In another exemplary embodiment of the invention, the channel 11 can be a wireless connection for communication and a battery for power. Yet in another exemplary embodiment of the invention, the communication channel and power channel 11 is a 4-way audio jack connector, in which a stereo earphone line and a microphone line are available. The microphone line is used for communicating data from the assembly 15 to the device and earphones line is used for harvesting/receiving power from the device which the assembly 15 is connected to.

In the fourth embodiment of the invention, the intelligent device with display 13 is connected to the assembly 15 through the communication and power channel 11. In an exemplary embodiment of the invention, the device 13 is a smartphone or tablet, whereas in another embodiment it is a laptop, desktop pc. This allows the algorithm implementing the method represented in FIG. 4 or FIG. 5 to be executed into the intelligent device. The acquired data regarding the frequency f and the current I are transmitted directly to the device 13 and then elaborated with the method in FIG. 4 or FIG. 5.

In the fifth embodiment of the invention, the vehicle interlock system 14 is connected to the assembly 15 through the communication and power channel 11. After performing the measurement and having it sent to the interlock system, the interlock system can decide whether to allow the user to ignite the vehicle or not, according to the data received.

In an exemplary embodiment of the invention, the device 14 is provided with an intelligent microprocessor system. This allows the algorithm implementing the method represented in FIG. 4 or FIG. 5 to be executed into the intelligent device. The acquired data regarding the frequency f and the current I are transmitted directly to the device 14 and then elaborated with the method in FIG. 4 or FIG. 5.

Referring to FIG. 4, the implemented method makes the device to be in the START condition 51 after powering it on, either by means of a power switch or by connecting it to the devices 13 or 14. From this condition, the system passes to the condition 52 for a determined amount of time (called the sampling window), in which it samples the sampler data, in form of the detected frequency f. The amount of time spent in each sampling window depends on the resolution with which the condition 53 has to be evaluated. In the present device, sampling windows between 50 ms and 500 ms can be used. The frequency f is converted to the flow rate F inverting equation (1). Then the system passes to the decisional condition 53 in which, if the flow F is greater than a predefined threshold F₂, the system passes to the condition 54, otherwise it goes back to the condition 52. In the condition 54, both the vortex flow-meter sampler and the sensor are sampled for the time of the sampling window. Then the system passes to the condition 55 in which the acquired flow data is integrated to obtain the total volume data according to the equation (2).

V(nT)=Eₖ₌₁ⁿF(kT)·T (2)

In which F(kT) is the flow value recovered with equation (1) from the frequency f measured in the k-th sampling window, T is the length of the sampling window, V(nT) is the volume summed from the first to the n-th sampling window, and the first sampling window is taken when the condition 53 is first verified, and so determines the beginning of the flow.

Then the system passes to the decisional condition 56 in which, if the volume V is greater than a predefined threshold V₁, the system passes to the condition 58, otherwise it goes to the condition 57. In this decisional condition, if the flow F is greater than a predefined threshold F₁ the system goes back to the condition 54 in which it continues to sample and integrate the two values, otherwise it passes to the FAIL condition 62 in which the measurement has failed as the user stopped blowing and the sampled volume is not enough to perform the measurement.

In the condition 58, also the acquired sensor current I is integrated to obtain the total charge Q thanks to the equation (3).

Q(nT)=Eₖ₌₁ⁿI(kT)·T (3)

In which I(kT) is the sensor current measured and optionally averaged in the k-th sampling window, T is the length of the sampling window, Q(nT) is the charge summed from the first to the n-th sampling window, and the first sampling window is taken when the condition 53 is first verified, and so determines the beginning of the flow.

The system then passes to the condition 59 in which the BrAC is calculated on the basis of the acquired data, for example, in one exemplary embodiment of the invention the BrAC is obtained with equation (4), which accounts for the count of ethyl alcohol quantity in the given volume.

$$BrAC = K \cdot \frac{Q}{V} \qquad ( 4 )$$

In which K is a calibration parameter, Q the charge obtained from equation (3) and V is the volume obtained from equation (2). The system then passes to the condition 60 in which the BrAC result is displayed, or transmitted or stored for further usage. The measurement process then concludes to the END 61. In an exemplary embodiment of the invention, a temperature sensor is added to the system and equation (4) is improved according to the variation of the sensor conversion efficiency to operating temperature, resulting in equation (5).

$$BrAC = K ( {T}_{sens} ) \cdot \frac{Q}{V} \qquad ( 5 )$$

wherein K(Tₛₑₙₛ) is a function of the sensor temperature Tₛₑₙₛ and K(Tₛₑₙₛ) is obtained by interpolation of calibration points measured at different temperatures;

In an exemplary embodiment of the invention, together with the BrAC, also the BAC is displayed at the end of the measurement, and is calculated thanks to the well-known relation (6)

BAC=K′·BrAC (6)

In which K′ is a constant which varies from 2100 to 2300, depending on the subject and on the drinking time.

In another embodiment of the invention, after terminating the execution, the system goes back to the START condition 51 for a new measurement.

In the following, each time the flow value F is mentioned, it is implicitly meant that the sampler data is acquired for the above-mentioned sampling window and the flow F is recovered from the measured frequency f.

Typical values considered for the thresholds are: F₁=10 L/min, F₂=11 L/min, V₁=1.5 L.

Referring to FIG. 5, the implemented method makes the device to be in the RECOVERY condition 71 after powering it on, either by means of a power switch or by connecting it to the devices 13 or 14. From this condition, when the sensor current I is less than a determined constant I_{rec} the system passes to the STDBY status 72. From this condition, if the sensor current I goes higher than the constant I_{det} the system passes to the ERROR TOOSOFT status 73, because it is likely that a weak breath was sufficient to determine a sensor current but not a flow measurement.

From the STDBY status 72, if the detected flow is greater than Fₕᵢ, the system passes to the FLOW DETECTED status 75. In this condition, flow and current are integrated according to equations (2) and (3). From this condition, if the flow remains greater than Fₗₒ for a time T_{det}, the system passes to the FLOW CONFIRMED status 76 in which the integration is continued, or, in case the detected flow is less than Fₗₒ, the system goes back to the STDBY status 72. From the FLOW CONFIRMED status 76, if the detected flow is greater than Fₗₒ and the sensor current is less than to I_{det} the system passes to the ERROR NOBAC status 74, which means that either the flow ethanol concentration is less than value associated to I_{det}, or that the measured flow was due not to an actual flow but to an interference; or to the FLOWBLOWING status 78, in which the integration is continued. From the ERROR NOBAC status 74, after the time Tₑᵣᵣ, the system terminates the execution. Alternatively, if also the current is above I_{det}, the system recognizes actual flow and goes on with the measurement process, which means that the user is breathing inside the device. From the FLOW BLOWING status 78, the system passes to the BLOW PAUSE status 77 if the flow is less than Fₗₒ, as the user could stop blowing for a while; or to the state FLOW ENOUGH if the integrated volume V is greater than V₀, which means that the blown volume is sufficient to perform the measurement. In the FLOW ENOUGH state 80 the integration is continued. From the BLOW PAUSE status 77, the system goes back to the FLOW BLOWING status 78 if the flow is greater than Fₗₒ, or to the ERROR N_ENOUGH status 79 if the flow remains lower than Fₗₒ for at least the time Tₚₐᵤₛₑ, which means that the blown volume is not enough to perform the measurement. From the FLOW ENOUGH status 80, the system passes to the DAQ state 81 if the flow is lower than Fₗₒ. In this condition, the system has received a sufficient and acceptable amount of volume and is waiting for the sensor current I to terminate its transient. From the ERROR N_ENOUGH status 79, after the time Tₑᵣᵣ, the system terminates the execution. From the DAQ status 81, the system passes to the DONE status 83 if the current becomes lower than I_{det}, or to the BOUNCE status 82 if the current is increasing after a timeout T_{decay} from when the system passes for the first time to the DAQ status 81. This condition is provided to tolerate the decay response time and little current peaks over a general decay behaviour. From the BOUNCE status 82 the system passes to the ERROR REBLOW status 84 if the current is increasing for a period greater than T_{bounce}, or back to the DAQ status 81 if the current is increasing. In the DONE status 83 the BAC and/or the BrAC are calculated according to the equation (4) or (5) and (6) and after the time T_{done}, the system terminates the execution. From the ERROR REBLOW status 84, after the time Tₑᵣᵣ, the system terminates the execution.

In another embodiment of the invention, after terminating the execution, the system goes back to the RECOVERY status 71 for a new measurement.

The aforementioned parameters are:
F(tᵢ) is the flow value measured in the sampling window tᵢ, Δtᵢ is the length of the sampling window tᵢ;
I(tᵢ) is the sensor current measured and optionally averaged in the sampling window tᵢ;
V(tk) is the volume summed from the instant t₀ to the instant tₖ;
Fₗₒ is an appropriate threshold for the flow to establish that the detected flow is sufficient to begin the measurement, i.e. 10 L/min;
Fₕᵢ is an appropriate threshold for the flow to establish whether the flow remains sufficient to continue the measurement, i.e. 11 L/min;
I_{rec} is an appropriate threshold for the current to establish that the sensor functionality is recovered, i.e. 15 μA;
I_{det} is an appropriate threshold for the current to establish that a minimum alcohol presence is detected, i.e. 25 μA;
V₀ is an appropriate threshold for the volume to establish that a sufficient volume has been sampled to perform the measurement, i.e. 2 L;
Tₚₐᵤₛₑ is an appropriate timeout to allow the user to stop breathing for a while, i.e. 0.5 s;
T_{det} is an appropriate timeout to establish whether a sufficient flow is been blown constantly or impulsively, i.e. 2 s;
T_{decay} is an appropriate timeout to allow the sensor current not to begin decaying right after the flow stops, i.e. 2 s;
T_{bounce} is an appropriate timeout to filter sensor current bounces during decay, i.e. 0.2 s;
T_{done} is an appropriate timeout to display result and after return to recovery status, i.e. 3 s;
Tₑᵣᵣ is an appropriate timeout to display error and after return to recovery status, i.e. 3 s:

The Fₕᵢ and Fₗₒ threshold values are generally different in order to grant a hysteretic behaviour.

The implemented method allows accounting for device misuse such as intermittent flow, weak flow, repeated or too strong flow, that could seriously affect both accuracy and precision of the measurements.

By the term “appropriate”, it is intended that the appropriate values can be set by the manufacturer in order to bring the device in compliance with the local laws concerning the ethanol levels (BAC) in order to determine a person's sobriety. In the specific embodiment of the present invention concerning a vehicle interlock system, the appropriate values can be set in order to determine start of the vehicle or any other means allowing or not the operation of the vehicle.

In other embodiments of the present invention, the appropriate values, when referring to specific gaseous substances to be measured in a person's breath, can be set in order to comply for predetermined levels of said substance, for example referring to clinical parameters.

What is claimed:

1. An apparatus for detecting gas concentration in exhaled breath of a subject, comprising a sampling device, a detector and a display device, wherein the sampling device is a vortex flow-meter.

2. The apparatus according to claim 1, wherein the apparatus detects ethanol gas concentration in exhaled breath and the display device displays the ethanol blood concentration detected from the subject.

3. The apparatus according to claim 2, wherein the detected ethanol concentration in the exhaled breath correlates to ethanol blood concentration in the subject.

4. The apparatus according to claim 1, wherein said detector further comprises a fuel-cell.

5. The apparatus according to claim 2, wherein the apparatus comprises
a. an assembly comprising a main breath channel for breath sampling, said main breath channel connected in parallel with a secondary channel so that a portion of the main flow of the sampled breath passes through the secondary channel and returns to the main channel, or an assembly comprising a main breath channel for breath sampling, said main channel connected in series with a vortex flow-meter, a first tangential pipe connected to the vortex flow-meter and to a sensor, and connected to the vortex flow-meter by a second tangential pipe;
b. an ethanol sensor in the secondary channel, configured that the portion of the sampled breath passes through said sensor; said sensor optionally comprising a temperature sensor;
c. a vortex flow-meter placed at the end of the main channel to collect the sampled breath;
d. a transducer located in the vortex flow-meter to obtain an electrical signal;
e. two amplifiers connected by way of input to the sensor and to the transducer, respectively and by way of output to a microprocessor with a memory containing a software algorithm for the calculation of ethyl alcohol concentration; and
f. a display connected to the microprocessor.

6. The apparatus according to claim 5, wherein in the apparatus comprises
a. an assembly comprising a main breath channel for breath sampling, said main channel connected in series with a vortex flow-meter, a first tangential pipe connected to the vortex flow-meter and to a sensor, and connected to the vortex flow-meter by a second tangential pipe;
b. an ethanol sensor in the secondary channel, configured that the portion of the sampled breath passes through said sensor; said sensor optionally comprising a temperature sensor;
c. a vortex flow-meter placed at the end of the main channel to collect the sampled breath;
d. a transducer located in the vortex flow-meter to obtain an electrical signal;
e. two amplifiers connected by way of input to the sensor and to the transducer, respectively and by way of output to a microprocessor with a memory containing a software algorithm for the calculation of ethyl alcohol concentration; and
f. a display connected to the microprocessor.

7. The apparatus according to claim 5, wherein said transducer comprises a microphone or a piezoelectric transducer.

8. The apparatus according to claim 5, wherein said vortex flow-meter has the following dimensions: Dᵢₙ from 4 to 7 mm, Dₒᵤₜ from 6 to 9 mm, D_{c} from 25 to 35 mm; and Lₒᵤₜ from 4 to 1 5 mm; wherein Dᵢₙ represents the diameter of the tangential inlet, Dₒᵤₜ represents the diameter of the axial outlet, D_{c} represents the diameter of the chamber and Lₒᵤₜ represents the length of the is axial outlet.

9. The apparatus according to claim 5, wherein the apparatus comprises at least one battery or a power channel for connecting to an external power source.

10. The apparatus according to claim 9, wherein the power channel comprises a communication channel and a power channel, or only a communication channel.

11. The apparatus according to claim 10, wherein the communication and power channel comprises a Universal Serial Bus (USB) connection.

12. The apparatus according to claim 10, wherein the communication channel comprises a wireless transceiver and a battery.

13. The apparatus according to claim 10, wherein the communication channel comprises an audio jack connector and a battery.

14. The apparatus according to claim 10, wherein the communication channel and power channel comprise an audio jack connector.

15. The apparatus according to claim 14, wherein the apparatus further comprises an intelligent device having a display connected to the microprocessor.

16. The apparatus according to claim 15, wherein the intelligent device is selected from the group consisting of a smartphone, a laptop and a desktop personal computer.

17. The apparatus according to claim 5, wherein the apparatus further comprises a vehicle interlock system connected to the microprocessor.

18. The apparatus according to claim 17, wherein the intelligent device or the vehicle interlock system is connected to the microprocessor via a data communication channel.

19. The apparatus according to claim 18, wherein the data communication channel comprises a wireless communication channel.

20. A method for detecting breath alcohol concentration (BrAC) of a subject, comprising sampling the subject's breath into the apparatus of claim 2, thereby detecting the BrAC of the subject.
